SEQUENCE LISTING (4 sequences)

SEQ ID NO: 1 - protein, 16 aa, Artificial Sequence
  Description of Artificial SequenceEpitopes of HIV Envelope
SLWNWFNITNWLWYIK

SEQ ID NO: 2 - protein, 20 aa, Artificial Sequence
  Description of Artificial Sequence Epitopes of HIV Envelope
QQEKNEQELLELDKWASLWN

SEQ ID NO: 3 - protein, 610 aa, Artificial Sequence
  Description of Artificial SequenceConsensus of HIV Envelope Protein
MRVRGIQRNCQHLWRWGTLILGMLMICSAAENLWVTVYYGVPVWKEANTTLFCASDAKAYDTEVHNVWATHACVPTDPNPQEIVLENVTENFNMWKNNMVEQMHEDIISLWDQSLKPCVKLTPLCVTLNCTNVNVTNTTNNTEEKGEIKNCSFNITTEIRDKKQKVYALFYRLDVVPIDDNNNNSSNYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQAFYATGDIIGDIRQAHCNISGTKWNKTLQQVAKKLREHFNNKTIIFKPSSGGDLEITTHSFNCRGEFFYCNTSGLFNSTWIGNGTKNNNNTNDTITLPCRIKQIINMWQGVGQAMYAPPIEGKITCKSNITGLLLTRDGGNNNTNETEIFRPGGGDMRDNWRSELYKYKVVKIEPLGVAPTKAKLTVQARQLLSGIVQQQSNLLRAIEAQQHLLQLTVWGIKQLQARVLAVERYLKDQQLEIWDNMTWMEWEREINNYTDIIYSLIEESQNQQEKNEQELLALDKWASLWNWFDITNWLW

SEQ ID NO: 4 - DNA, 1839 bp, Artificial Sequence
  Description of Artificial Sequence HIV Envelope Encoding Sequence
atgcgcgtgcgcggcatccagcgcaactgccagcacctgtggcgctggggcaccctgatcctgggcatgctgatgatctgctccgccgccgagaacctgtgggtgaccgtgtactacggcgtgcccgtgtggaaggaggccaacaccaccctgttctgcgcctccgacgccaaggcctacgacaccgaggtgcacaacgtgtgggccacccacgcctgcgtgcccaccgaccccaacccccaggagatcgtgctggagaacgtgaccgagaacttcaacatgtggaagaacaacatggtggagcagatgcacgaggacatcatctccctgtgggaccagtccctgaagccctgcgtgaagctgacccccctgtgcgtgaccctgaactgcaccaacgtgaacgtgaccaacaccaccaacaacaccgaggagaagggcgagatcaagaactgctccttcaacatcaccaccgagatccgcgacaagaagcagaaggtgtacgccctgttctaccgcctggacgtggtgcccatcgacgacaacaacaacaactcctccaactaccgcctgatcaactgcaacacctccgccatcacccaggcctgccccaaggtgtccttcgagcccatccccatccactactgcgcccccgccggcttcgccatcctgaagtgcaacgacaagaagttcaacggcaccggcccctgcaagaacgtgtccaccgtgcagtgcacccacggcatcaagcccgtggtgtccacccagctgctgctgaacggctccctggccgaggaggagatcatcatccgctccgagaacatcaccaacaacgccaagaccatcatcgtgcagctgaacgagtccgtggagatcaactgcacccgccccaacaacaacacccgcaagtccatccgcatcggccccggccaggccttctacgccaccggcgacatcatcggcgacatccgccaggcccactgcaacatctccggcaccaagtggaacaagaccctgcagcaggtggccaagaagctgcgcgagcacttcaacaacaagaccatcatcttcaagccctcctccggcggcgacctggagatcaccacccactccttcaactgccgcggcgagttcttctactgcaacacctccggcctgttcaactccacctggatcggcaacggcaccaagaacaacaacaacaccaacgacaccatcaccctgccctgccgcatcaagcagatcatcaacatgtggcagggcgtgggccaggccatgtacgccccccccatcgagggcaagatcacctgcaagtccaacatcaccggcctgctgctgacccgcgacggcggcaacaacaacaccaacgagaccgagatcttccgccccggcggcggcgacatgcgcgacaactggcgctccgagctgtacaagtacaaggtggtgaagatcgagcccctgggcgtggcccccaccaaggccaagcttaccgtgcaggcccgccagctgctgtccggcatcgtgcagcagcagtccaacctgctgcgcgccatcgaggcccagcagcacctgctgcagctgaccgtgtggggcatcaagcagctgcaggcccgcgtgctggccgtggagcgctacctgaaggaccagcagctcgagatctgggacaacatgacctggatggagtgggagcgcgagatcaacaactacaccgacatcatctactccctgatcgaggagtcccagaaccagcaggagaagaacgagcaggagctgctggccctggacaagtgggcctccctgtggaactggttcgacatcaccaactggctgtggtgaggatcc

(19) United States
(12) Patent Application Publication
(10) Pub. No.: US 2007/0086946 A1
(43) Pub. Date: Apr. 19, 2007

(54) Assay

(75) Inventor: Barton F. Haynes, Durham, NC (US)

Correspondence Address: NIXON & VANDERHYE, PC, 901 NORTH GLEBE ROAD, 11TH FLOOR, ARLINGTON, VA, 22203, US

(73) Assignee: DUKE UNIVERSITY, Durham, NC (US)

(21) Appl. No.: 11/516,665

(22) Filed: Sep. 7, 2006

Related U.S. Application Data
(60) Provisional application No. 60/714,334, filed on Sep. 7, 2005.

(51) Int. Cl. A61K 49/00; C12Q 1/70; A01K 67/027

(52) U.S. Cl. 424/9.2; 435/5; 800/10

(57) ABSTRACT

The present invention relates, in general, to a method of assaying an immune response induced by an immunogen, and, more particularly, to a method of assaying a immunogen for its ability to induce a desired immune response, wherein the assay is effected in an autoimmune animal.

This application claims priority from U.S. Provisional Application No. 60/714,334, filed Sep. 7, 2005, the entire content of which is incorporated herein by reference.

This invention was made with government support under AI52816 awarded by the National Institutes of Health. The government has certain rights in the invention.

### TECHNICAL FIELD

The present invention relates, in general, to a method of assaying an immune response induced by an immunogen, and, more particularly, to a method of assaying a immunogen for its ability to induce a desired immune response, wherein the assay is effected in an autoimmune animal.

### BACKGROUND OF THE INVENTION

Several fundamental breakthroughs were needed to enable polio vaccine developers to make rapid progress in development of a polio vaccine. Jonas Salk performed the tedious work and determined the three types of polio virus, and realized that one needed to make a vaccine against all three strains for the vaccine to be effective. John Enders discovered how to grow the polio virus in vitro and that opened the way for rapid assessment of vaccine candidates and production of the killed and attenuated polio vaccines. A major question facing HIV-1 vaccine researchers is, “Why are broadly reactive neutralizing antibodies not made in acute or early infection, why are they rarely made in chronic disease, and why are they not made in response to vaccination with HIV-1 envelope?” The majority of attention to these questions has been devoted to studies of the viral envelope and not the host immune response. Autologous, strain-specific neutralizing antibodies (Nabs) are routinely made early in primary infection; they generally target exposed variable loop epitopes including those present on V1 V2, V3, and possibly V4, and virus escape from neutralization is rapid (Wei et al, Nature 422 (6929), 307-12 (2003); Richman et al., 2003). Antibody responses to CD4 or co-receptor binding surfaces have been documented but, except for the CD4bs mAb IgG1b12, such antibodies generally have weak neutralizing potency (Burton et al, Nature Immunology 5(3), 233-6 (2004)). The four defined epitopes on HIV-1 envelope to which rare broadly reactive Nabs bind are thus the CD4 binding site (CD4BS) (mAb IgG1b12) (Zwick et al, J. Virology 77(10), 5863-76 (2003)); the membrane proximal external region (MPER) epitopes defined by human mAbs 2F5 and 4E10 (Scanlan et al, Adv. Exper. Med. Biol. 535, 205-18)(2003); Armbruster et al., J. Antimicro. Chem. 54, 915-92.0 (2004); Zwick et al, J. Virology 79, 1252-1261 (2005)); and the glycan epitope defined by mAb 2G12 (Scanlan et al, Adv. Exper. Med. Biol. 535, 205-18 (2003)). These mAbs are all unusual: two are IgG3 (2F5 and 4E10), one has a unique Ig dimer structure (2G12), and one has a very hydrophobic CDR3 (2F5). Moreover, all four have unusually long CDR3 regions (Burton et al, Nature Immunology 5(3), 233-6 (2004); Kunert et al, AIDS Res. Hum. Retro. 20(7), 755-62 (2004); Zwick et al, J. Virology 78(6), 3155-61 (2004), and three of the four mAbs (2F5, 4E10 and IgG1b12) have recently been found to be autoreactive (Haynes et al, Science 308:1906-1908 (2005)).

What is needed in HIV vaccine research, and for many other vaccine development efforts, is enabling technology in the form of an assay that makes it possible to determine whether the correct structures are present in or on a vaccine candidate (that is, whether the immunogen is adequate) and further makes it possible to determine whether the failure of an adequate immunogen to induce antibodies against a desired region is because the host immune system is not making the desired response. Such an assay would allow vaccine developers to focus on the formulation of the immunogen, for example, in optimal adjuvants, instead of only focusing on modifying the structure of the vaccine when the structure is, in fact, not the problem.

A major reason why the immune system does not respond to vaccine immunogens (that is, adequate immunogens) is that the epitopes on the immunogen are either mimics of self antigens, or are self antigens, and thus induce B cell tolerance by B cell deletion or negative selection and/or by B cell receptor editing mechanisms, all targeted at decreasing the autoreactivity of a B cell response, and making the resultant antibody less autoreactive and more monospecific for the vaccine.

The present invention provides an assay that makes it possible to determine whether the non-immunogenicity of structurally correct epitopes results from the fact that such epitopes induce polyspecific autoreactive antibodies that are either deleted by immune tolerance, B cell apoptosis (negative selection) or receptor editing.

### SUMMARY OF THE INVENTION

The present invention relates generally to a method of assaying an immune response. More specifically, the invention relates to a method of assaying a immunogen for its ability to induce a desired immune response, wherein the assay is effected in an autoimmune animal.

### BRIEF DESCRIPTION OF THE DRAWINGS

FIGS. 1A and 1B. FIG. 1A. Monomeric nature of the gp120 protein. 1B. Oligomeric nature of the gp140 Envs tested.

FIG. 2. Antibodies to the 2F5 gp41 epitope in normal BALB/c mice immunized with HIV-1 gp140 Env oligomer.

FIG. 3. Antibodies to the 2F5 gp41 epitope in autoimmune MRL/lpr^{-/-} mice immunized with HIV-1 gp140 Env oligomer.

FIGS. 4A and 4B. Year 2001 group M consensus envelope protein (CON-S) (4A) and encoding sequence (4B).

FIG. 5. Reactivity of serum from naïve BALB/C mice to cardiolipin and Env antigens.

FIG. 6. Reactivity of serum from naïve MRL/lpr^{-/-} to cardiolipin and Env antigens.

FIG. 7. B cell tetramers.

FIG. 8. Crosslinking of B cell Ig receptors.

FIG. 9. 2F5 tetramer binding to splenic B cell populations in naïve BALB/C and MRL mice.

FIG. 10. 2F5 tetramer binds to distinct splenic B cell subsets in naïve BALB/C and MRL mice.

### DETAILED DESCRIPTION OF THE INVENTION

The present invention relates to a rapid and simple screen for identification of, and distinguishing between, immune responses that are not made because of host immune control and down-regulation of such responses, and immune responses that are not made because of defects in the vaccine epitopes themselves. The instant screening methodology should speed up vaccine development, including but not limited to, development of an HIV vaccine.

The present invention results, at least in part, from studies designed to explore the evolution of neutralizing antibody responses to HIV-1 in acute and early infection. The studies involve mapping of the epitopes recognized by narrow and broadly reactive Nabs, and address the novel concept that molecular mimicry exists between certain Env epitopes on HIV-1, including the broadly reactive MPER 2F5 and 4E10 epitopes, and normal host antigens resulting in host B cell tolerance.

Recent data demonstrate that mAbs 2F5, 4E10 and IgG1b12 are polyspecific, autoreactive Abs that bind with high affinity to multiple human autoantigens (Haynes et al, Science 308:1906-1908 (2005)). These data thus suggest a new explanation and paradigm for understanding the ineffective host neutralizing antibody response to HIV-1 in both acute infection and in normal subjects following vaccination with HIV-1 envelope. Thus, HIV-1 may have evolved to escape antibody responses by having conserved neutralizing epitopes as mimics of autoantibody epitopes. These data suggest the hypothesis that acute HIV infection (AHI) and current HIV-1 vaccines do not routinely induce robust anti-envelope neutralizing antibodies because antibodies targeting conserved epitopes are derived from autoreactive B cell clones that are normally deleted or made tolerant upon antigenic stimulation by HIV-1 Env.

These observations may also explain the rare occurrence of HIV-1 in SLE patients who may be unable to delete these self-reactive clones (Fox and Isenberg Arthritis and Rheum. 40: 1168, 1997; Palacios and Santos J. STD AIDS 15: 277, 2004). If broadly Nabs to HIV-1 are made in the context of disordered B cell immunoregulation in autoimmune disease, then autoimmune patients may be fully or partially protected on this basis. Since the autoantigens recognized by humans are conserved throughout phylogeny, i.e., are also the same autoantigens recognized by mouse strains that have autoimmune disease, then it should be possible to explore the immunogenicity of epitopes on vaccines that may mimic autoantigens in animal models of autoimmune disease for their immunogenicity.

The present invention uses autoimmune animal (preferably rodent, more preferably mouse) strains to screen immunogens for being subjected to toleragenic host B cell regulatory mechanisms. In accordance with a preferred embodiment of the invention, an immunogen is injected into both normal mice and autoimmune mice that have defects in B cell tolerance mechanisms. Advantageously the autoimmune mice are the MRL/lpr^{-/-} (Jackson labs MRL/MpJ-faslpr/J No. 000485) strain that has a mutation in the fas gene (CD95) that mediates programmed cell death in B cells (MRL/lpr^{-/-} mice represent a spontaneous model of SLE that closely resembles the human disease including polygenic inheritance, glomerulonephritis and gender bias). Antibodies against the epitope that one desires to induce antibodies to are measured in the serum after several immunizations in, for example, either ELISA or surface plasmon reasonance or in a functional antibody assay such as a HIV neutralizing antibody assay. When the immunogen is structurally correct and the desired immune responses are not made because of host control, then the normal mice do not respond and the autoimmune mice that have defects in B cell tolerance mechanisms do respond. When the immunogen is not structurally correct, or for whatever reason the desired epitope is not on the surface of the immunogen, then neither the normal mouse strain nor the autoimmune mouse strain respond.

For the 2F5 membrane proximal external region (MPER) of the HIV-1 gp160 envelope, that is a target of broadly neutralizing antibodies, the MRL/lpr^{-/-} strain provided this answer. That the MRL strain could responded to this MPER gp160 region and the BALB/c mouse strain did not, suggesting that production of this antibody was regulated by B cell deletion mechanisms involving induction of B cell clone apoptosis that is controlled by the fas gene.

Other mouse strains with different defects in B cell tolerance, such as non-B cell deletional mechanisms (such as B cell anergy) and B cell receptor editing, can be used to screen for the mechanisms of responding to other epitopes on HIV-1 and epitopes on other infectious agent vaccines. For example, there are several models of mouse autoimmune disease each with different mechanisms of breaks in tolerance that leads to autoreactive immune responses.

In MRL/lpr^{+/+} mice, there is a predisposition to making autoantibodies by genes that are not understood, and in the strain with the fas mutation, MRL/lpr^{-/-} mice, severe autoimmune disease with uncontrolled lymphoproliferation occurs. FAS is a tumor necrosis factor (TNF)-like surface receptor on lymphoid cells that mediates apoptosis when it encounters its ligand, Fas-L (Watanabe-Fukunaga et al, Nature 356: 314-317, 1992). A similar model from mutations in the fas ligand is found in the gld/gld^{-/-} mouse. NZB, NZW, and NZB/NZW F1 mice all have varying degrees of ability to make autoantibodies, with the F1 mice having frank and severe autoimmune disease similar to human lupus erythematosus (Rose and Mackay, The Autoimmune Diseases 3^{rd} Ed. Academic Press, NY, N.Y. 1998 p290-292). The Palmerston North strain of mice also make autoantibodies to self and have lupus like arthritis in older mice (Rose and Mackay, The Autoimmune Diseases 3^{rd} Ed. Academic Press, NY, N.Y. 1998 p290-292). The BXSB mouse model is one in which the Y chromosome associated autoimmunity accelerator (yaa) gene in the BXSB model that results in early death in males from glomerulonephritis related to high titers of anti-dsDNA autoantibodies (Rose and Mackay, The Autoimmune Diseases 3^{rd} Ed. Academic Press, NY, N.Y. 1998 p290-292).

As other genes are identified, there will be ever increasing numbers of potentially useful mouse strains produced from the process of homologous recombination or “knock-out” mouse technology (Smithies, O, Nat. Rev. Genet. 2005 May;6(5):419-25).

Underexpression of the following genes have been associated with autoimmune or uncontrolled lymphocyte growth in animals and humans: TNF alpha, IL-1 receptor antagonist, STAT-3, TGF beta, programmed death-1 (PD-1), Cytotoxic T lymphocyte antigen, 4 (CTLA-4), IL-10, Complement deficiency of C1, C2, C3 or C4, TNF factor receptor 1, Fas (CD95, apo1), Fas ligand, perforin, caspase 10, bcl-10, p53, bax, bcl-2, c-IAP2, and NAIP1 (Reviewed in Haynes and Fauci, Introduction to the Immune System Chapter 295 in Harrisons Principles in Internal Medicine 16^{th} Edition, 2005; McGraw Hill, NY, N.Y. Eds. Kasper, Brqawnwald, Fauci, Hauser, Longo, Jamison, from Table 295-12 of that edition). Mice with underexpression of knockouts of these genes are also suitable for use in the instant assay in the same manner as are the MRL mice for the 2F5 epitope of gp41 antibodies.

One hypothesis to explain why anti-MPER and IgG1b12-like antibodies are not routinely made by vaccinated animals and man, and by patients during AHI, is that the B cells making these species of antibodies are tolerant because they are suppressed by T regulatory cells naturally or induced by HIV-1. The loss of autoreactive B cell tolerance and induction of autoreactive antibody production may require both the generation of T cell help and overcoming suppression mediated by T regulatory cells. Recently, CD4+, CD25+ T cell number was studied over time in the NC cohort of AHI, and it was found that during the early stages of AHI (the first two months of infection before seropositivity) as the virus load falls, and CD4 and CD8 T cell proliferation wanes, the levels of circulating CD4+, CD25+ T cells rise (Sempowski et al J. Clin. Immunol. 25: 461-471, 2005).

T regulatory cells have been shown to control immune responses in a number of infections and clinical situations (Shevach, et al Immunol. Rev. 182: 58, 2001), and these cells can suppress the response to vaccines. Thus, mouse models in which T regulatory cells have been depleted or do not develop can be used to study which vaccine epitopes are not responded to because of host control by T regulatory cells (Tregs). For example, mice can be produced that do not develop T regulatory cells by neonatally thymectomizing them (reviewed in Shevach et al above). Mice can be depleted of CD4+CD25+Tregs using a depleting anti-CD25 antibody (FN Toka, S Suvas, and BT Rouse, J. Virol. 78:13082, 2004), using a depleting anti-CD25 immunotoxin, and by using a depleting anti-GITR antibody, since most Tregs are GITR+ at rest. Alternatively, T regulatory function can be inhibited in mice to provide a novel screening model by activation of GITR on CD4+ CD25+ Tregs using an agonistic antibody to GITR (Shmizu et al. Nat. Immunol. 3:135-42, 2005), or using an agonistic antibody to OX40, which is reported to ablate Treg immunosuppression but has other immunostimulatory effects on CD4+ and CD8+ T cells (Valzasina et al. Blood 105:2845-51, 2005). Alternatively, an agonistic antibody to 4-1BBL, which again is reported to ablate Treg immunosuppression but has other immunostimulatory effects on CD4+ and CD8+ T cells (Choi B K et al. J Leukoc Biol. 75:785-91, 2004) can be used, as can GITR ligand stimulation of GITR. Further, a novel animal model of lack of T regulatory cell development and survival, the CD7, CD28 double knock out mouse, can be used as described by Sempowski et al. (J. Immunol. 172: 787-794, 2004). This model of autoimmunity develops autoimmune thyroiditis.

All of the above-described manipulations can be used to create mice with defective T regulatory function that result in enhanced responses to vaccine immunizations and allow the discrimination of those immunogens that were inducing antibodies that are controlled by the host vs. those vaccine immunogens that are inherently non-immunogenic from the vaccine design point of view.

As regards animal models of receptor editing in the analysis of vaccine immunogens, there are two mouse strains of relevance here: 3-83 centrally deleting transgenic mice (Tiegs et al., JEM, 1993) and “macroself” transgenic mouse (Ait-Azzouzene et al., JEM, 2005). Both systems allow for sensitive measurement of receptor editing; the latter model has the advantage of assessing this in a normal, polyclonal immune system. An assessment can be made not only how but also where receptor editing is altered in macroself mice crossed onto autoimmune-prone strains such as MRL/lpr^{-/-} and BXSB. In these types of animal models, the effect of receptor editing on induction of the desired antibody types can be studied.

Immunogens that can be used in the context of the instant assay include, but are not limited to, immunogens from infectious agents such as HIV, Hepatitis C, Mycobacteria species, West Nile Virus, and Ebola Hemmorhagic Fever Virus. As regards HIV, the immunogen can be derived from, for example, HIV tat protein or HIV-1 envelope.

Certain aspects of the present invention are described in greater detail in the non-limiting Examples that follows.

### EXAMPLE 1

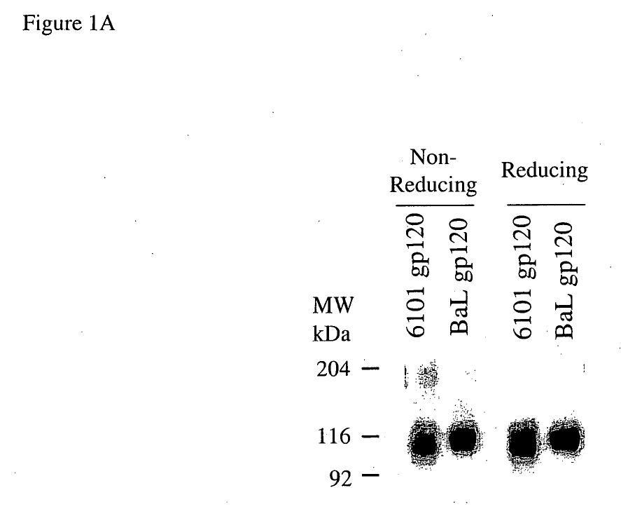

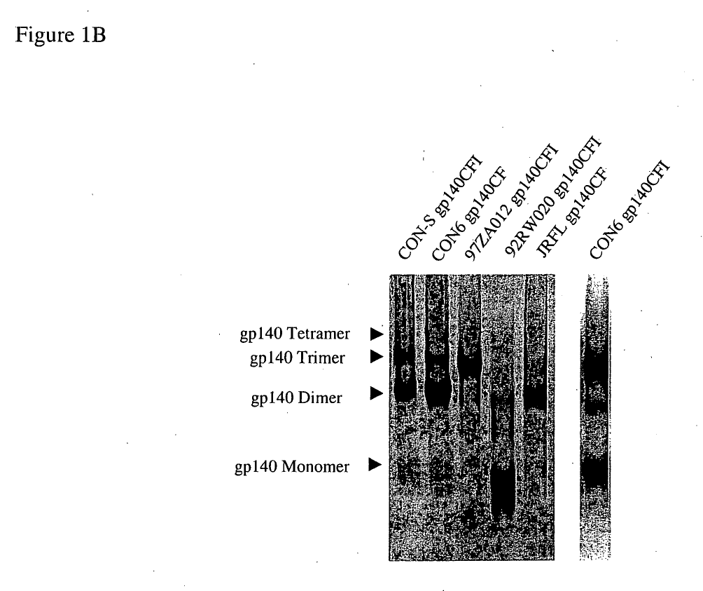

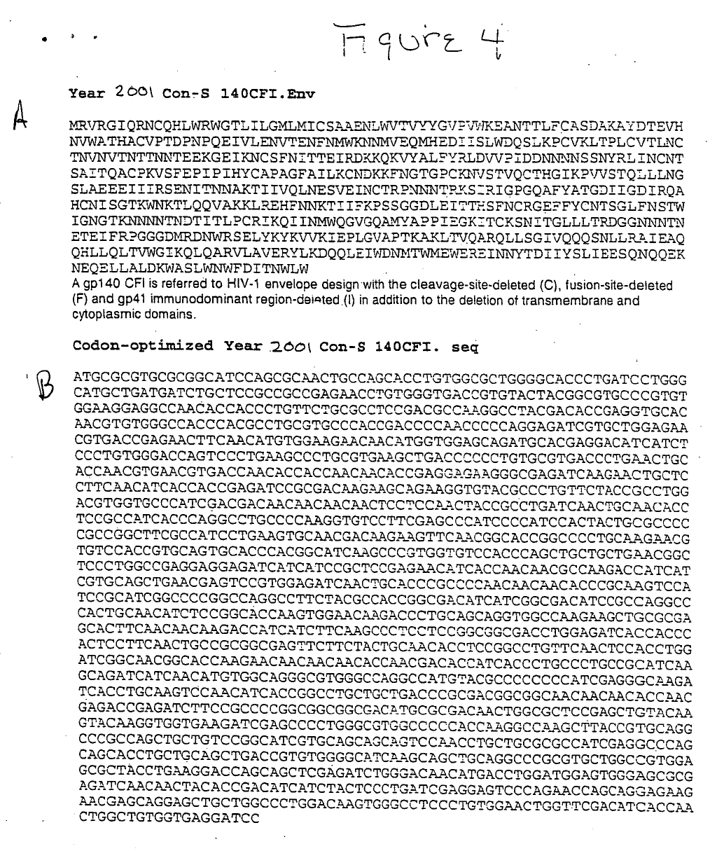

HIV-1 subtype C is the most common HIV-1 subtype in Africa and many parts of Asia. However, to date, HIV-1 vaccine candidate immunogens have not induced neutralizing antibodies against subtype C primary isolates of the desired potency and breadth. The centralized gene strategy has been used to overcome HIV-1 diversity and the year 2001 group M consensus envelope gene (CON-S) has been generated (see FIG. 4). CON-S Env has been compared with wild-type (WT) subtype A, B and C Envs for the ability to induce antibodies in guinea pigs that neutralize HIV-1 primary isolates. FIG. 1 shows the oligomeic nature of the gp140 Envs tested, and Table 1 shows the neutralizing antibody results. Envs that express the broadly neutralizing antibody epitopes of 2F5, 4E10, IgG1b12 and 2G12 like CON-S would be expected to induce antibodies that broadly neutralize HIV-1 while those that do not express these epitopes would not be expected to do so. While CON-S is the best of any known Env to date with regard to the ability to induce anti-subtype C neutralizing antibodies, the assays described herein define why production of more potent and more broadly neutralizing antibodies is not observed.

While WT A, B and C Envs all induced neutralization of select subtype B HIV-1 isolates, only subtype A Env neutralized any non-B isolates (TV-1, 92BR025.9, subtype Cs, and 92RW020, subtype A) (see Table 1). In contrast, the group M consensus CON-S gp140 ENV induced antibodies that neutralized the subtype B and A isolates that were neutralized by WT Env-induced antibodies, and as well, neutralized the subtype C isolates TV-1, DU123, ZM18108.6 and 92BR025. No mixture of subtype C or B Envs with CON-S augmented the breadth of CON-S induced-neutralizing antibodies. Absorption with V1-V5 CON-S peptides indicated that most of the neutralizing activity induced by CON-S gp140 was targeted primarily to the V3 loop. However, immunization with the CON-S V3 peptide itself could not induce similar antibodies. Thus, the year 2001 CON-S gp140 has a V3 loop that assumes a conformation in the context of the gp140 Env that induces antibodies that neutralize subsets of subtype B and C HIV-1 primary isolates with a breadth not seen in antibodies induced by WT Envs. Nonetheless, none of these Envs induced antibodies with specificities similar in breadth to those represented by broadly neutralizing mAbs 2F5, 4E10, 2G12 and IgG1b12.

Experimental Details

To determine the feasibility of using a mouse model to determine if there is host control over a desired vaccine epitope to target for an antibody response, the MRL/lpr^{-/-} mouse (Jackson labs 000485, MRL/MpJ-Fas lpr/J) was immunized with the Group M consensus oligomeric envelope, CON-S, 25 μg per mouse per immunization, formulated with Emulsigen in an oil in water emulsion per the manufacturer's recommendations. The control strain used was BALB/c mice from Charles River Laboratories. Animals were immunized on Day 0 and bled 10 days later.

Immunizations were performed as follows:

Con S in Emulsigen+oCpG: For use in mouse # 285, 286, 287, 288, 289; mouse # 295, 296, 297, 298, 299.

One batch of Con S 140 CFI in Emulsigen+oCpG is prepared that will serve to immunize all groups: 3 groups×5 mice/group=15 mice (add 2 extra mice)=17 mice.

Protein Needed: 25 μg Con S per mouse×17 mice=425 μg Con S

V_{final}=200 λ per mouse×17 mice=3400 λ.

Make 2×Emulsigen/oCpG solution: Volume=1700λ

10 μg oCpG/mouse×17 mice=170 μg oCpG=170λ at 1 mg/ml.

Emulsigen 20%=340λ

Saline=1190λ

Based on concentration of Con S 140 CFI, determine the volume needed for 425 μg of protein. Add saline to volume of 1.7 ml. Mix 1:1 with 1.7 ml of 2×Emulsigen/oCpG.

Inject each mouse with 200 μl, (100 μl×2 sites SC).

Schedule: Immunization #1+prebleed: day 0

Post-Immune 1 bleed: day 10

Oligo CpGs were made at the Duke DNA Synthesis facility using mouse CpG sequences from Pisetsky et al Clinical Immunology 100:157-163, 2001.

Super Block 2F5 Peptide Assay

Peptides are diluted to 2 μg/ml in 0.1M Sodium Bicarbonate. Coat wells of high-binding ELISA plate (Easywash, Costar 3369) with 100 μl/well at room temperature for 2 h, or overnight at 4° C.

Wash plate 3× with PBS-0.1% Tween 20.

Block wells 1 h with Super Block for 1 h, or overnight at 4° C.

Wash 2× with PBS-0.1% Tween 20.

Incubate 50 μl/well, diluted antibodies/sera in Super Block for 2 h at room temperature.

Wash plate 3× with PBS-0.1% Tween 20.

Add 100 μl/well of alkaline phosphatase conjugated, goat anti-mouse IgG (whole molecule, Sigma A-3562) antibody, diluted in Super Block, for 1 h at room temperature.

Wash plate 4× with PBS-0.1% Tween.

Add 100 μl/well of Substrate to wells for 45 minutes in the dark.

Read plates at OD 405.

Solutions for assay:

0.1M Sodium Bicarbonate

8.4 g in 1 L DI water

PBS-0.1% Tween20

1 ml Tween 20 in 1 L PBS

Super Block:

40 g Whey (obtained from James Robinson) (Whey is available from Ross Lab. (Columbus, Ohio) and Sigma-Aldrich (Cat #W1500)

150 ml Normal Goat Serum (Gibco, 16210-071)

5 ml Tween 20

In 1 L PBS

Substrate (per 100 ml), p-NPP (4-nitrophenyl phosphate di(2-amino-2-ethyl-1,3-propanediol) salt (Sigma, N6260)

1 mg/ml p-NPP in 50 mM carbonate/bicarbonate buffer, pH 9.6

10 mM MgCl₂.

The sequence of the P-4E10 and SP62 peptides is as follows:

P-4E10: SLWNWFNITNWLWYIK

SP62: QQEKNEQELLELDKWASLWN

### RESULTS

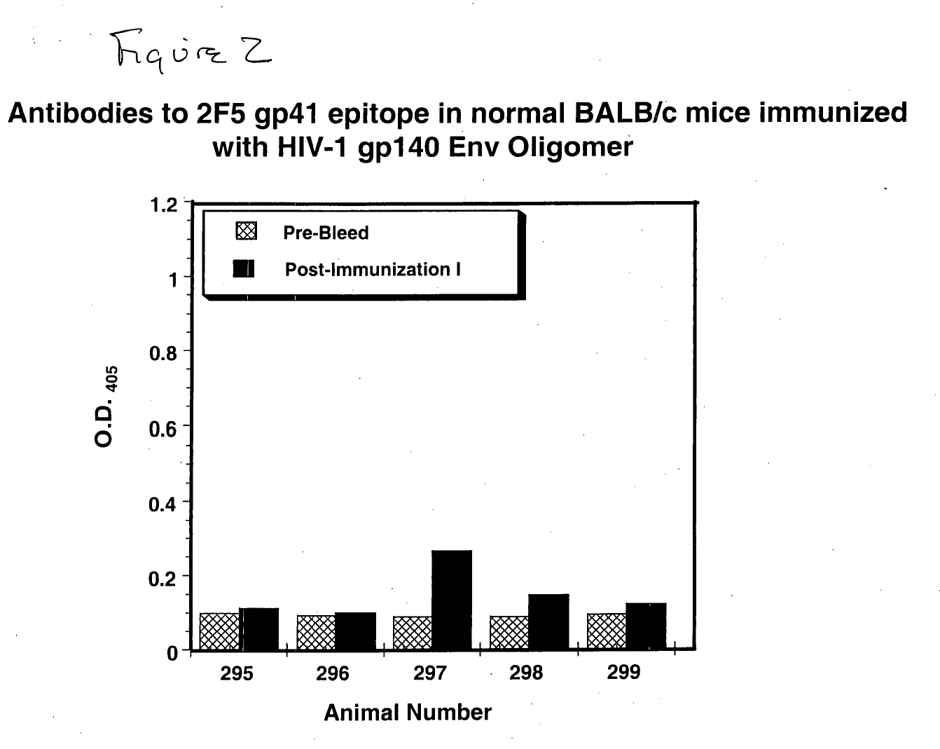

FIG. 2 shows that in BALB/c normal mice, CON-S after one immunization did not induce any significant antibody levels to the 2F5 gp41 peptide epitope. The hatched bars are the prebleed values for each of 5 mice, and the solid bars are the values after the first immunization.

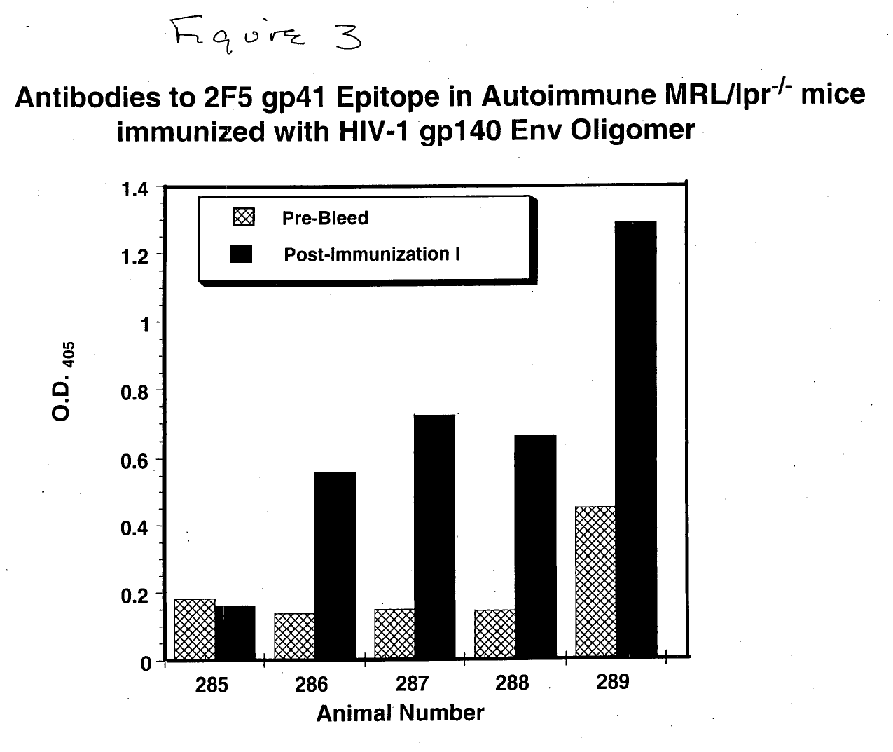

FIG. 3 shows that, in contrast to BALB/c mice, immunization of MRL/lpr^{-/-} mice with the CON-S oliogmer induced 4 of 5 animals to make high levels of antibodies against the 2F5 gp41 epitope. These data demonstrate that the mouse immune system can indeed recognize the 2F5 epitope on the surface of the Env oligomer, and that in the absence of the fas gene-mediated B cell negative selection by apoptosis, the animal can make (i.e., is released from B cell negative selection to make) an otherwise “forbidden” antibody response. These data indicate that there is nothing inherently wrong with the immunogen and that formulation of the immunogen in an adjuvant or other vector is required that will lead to breaking tolerance in otherwise normal animals. Since the mouse immune system can respond to this immunogen in this manner, in all likelihood the human immune system will respond in a similar manner.

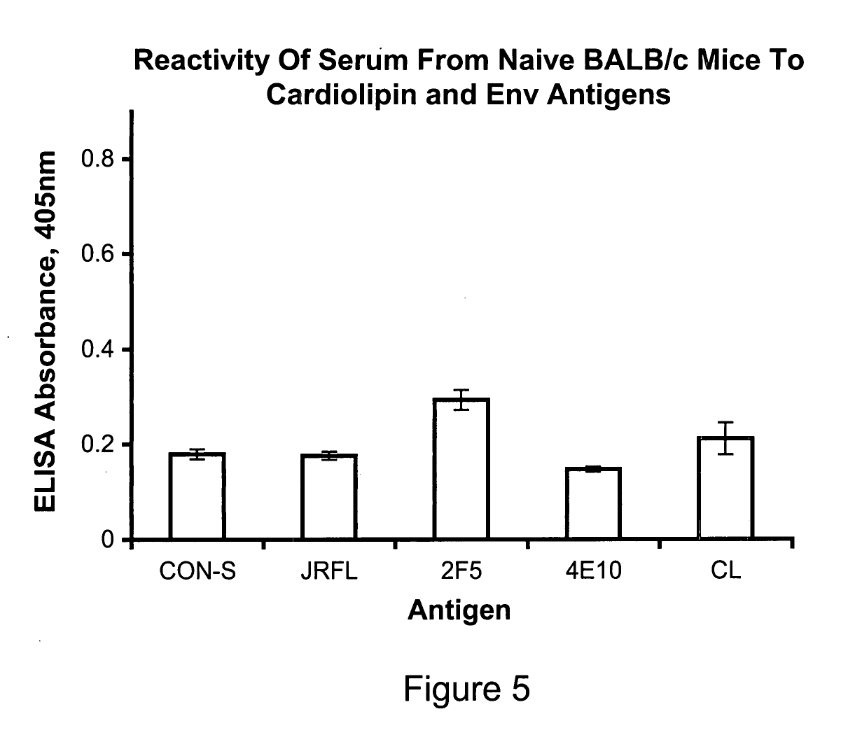

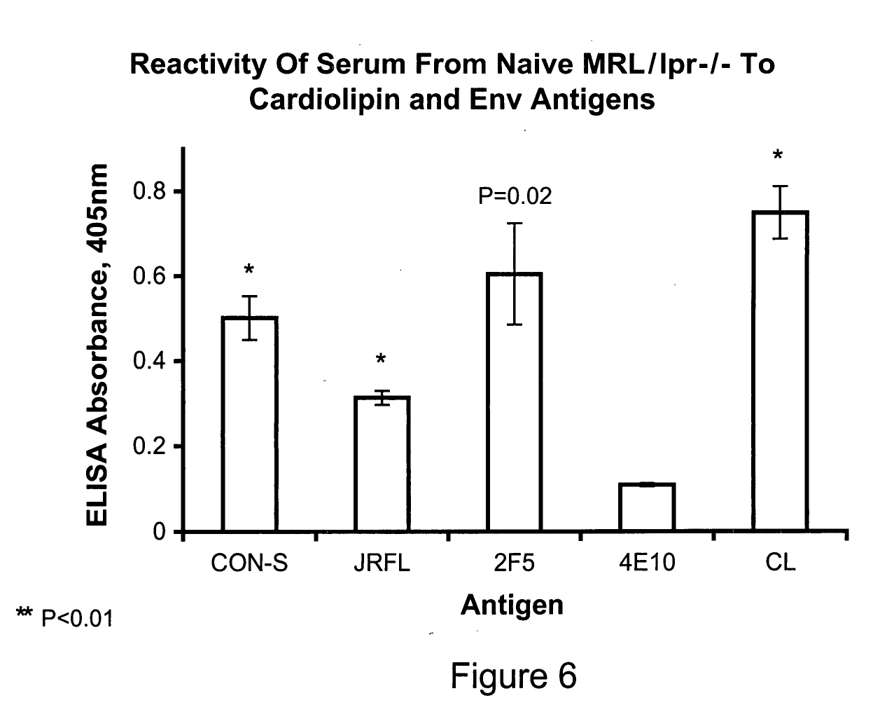

The data presented in FIG. 5 (BALB/C mice) and FIG. 6 (MRL/lpr^{-/-} mice) also show that MRL/lpr^{-/-} mice, in contrast to BALB/C mice, make high levels of antibodies against 2F5 and cardiolipin.

### EXAMPLE 2

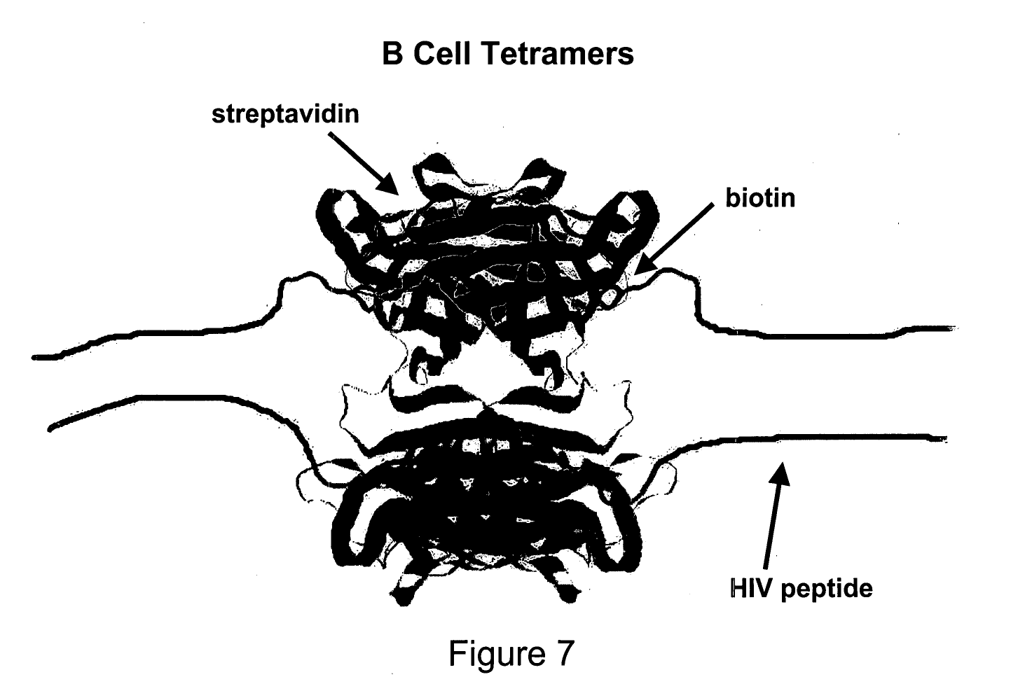

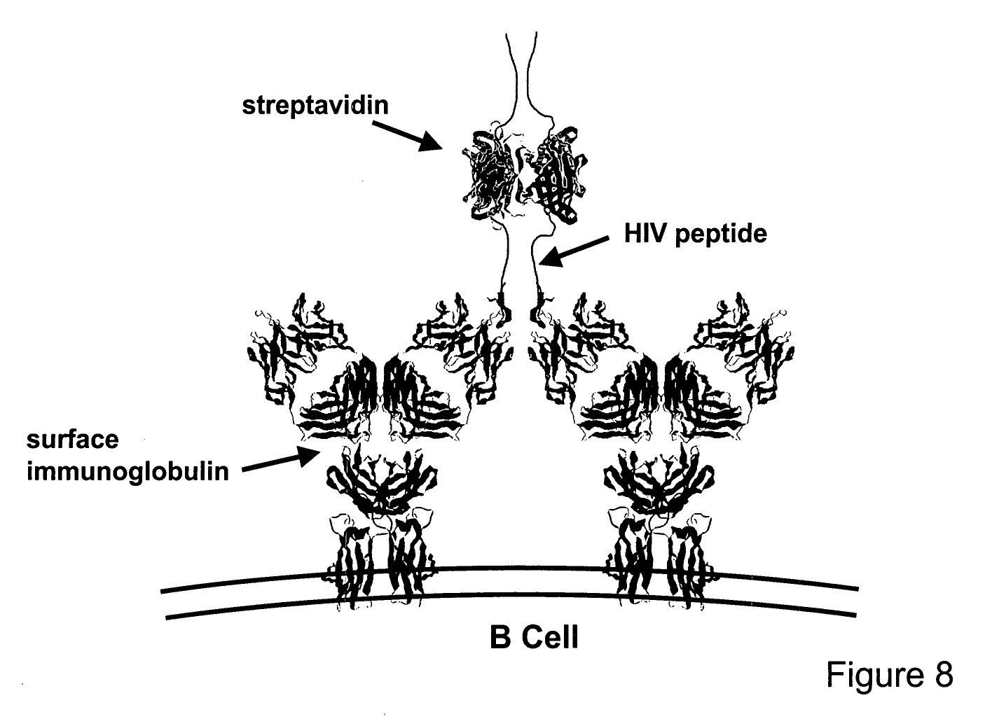

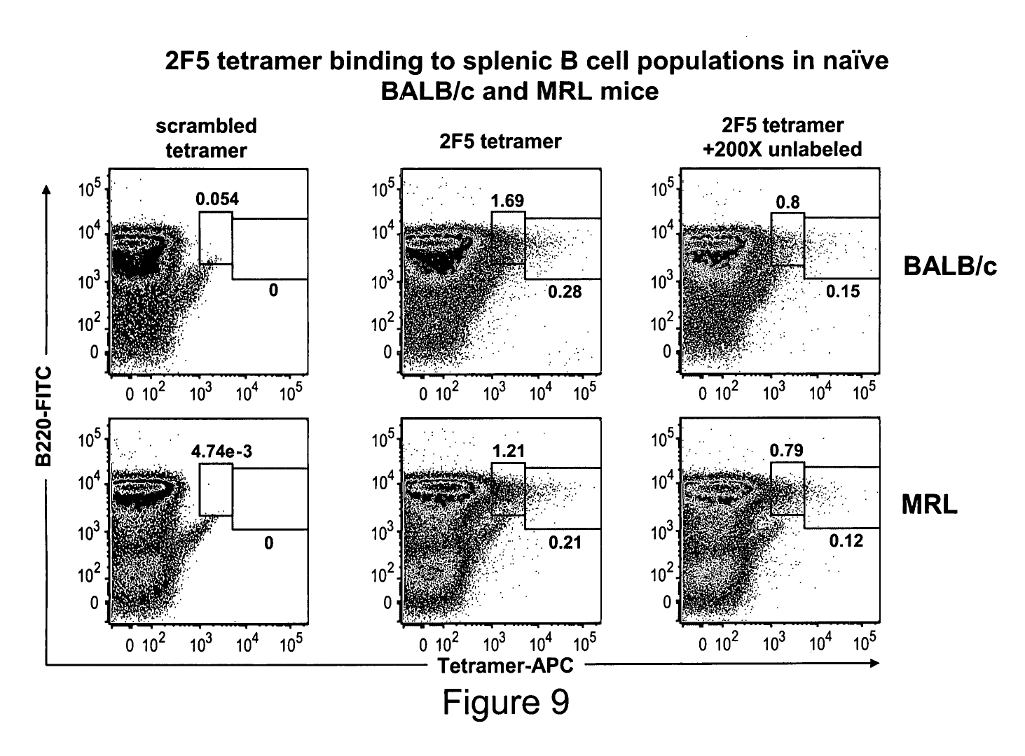

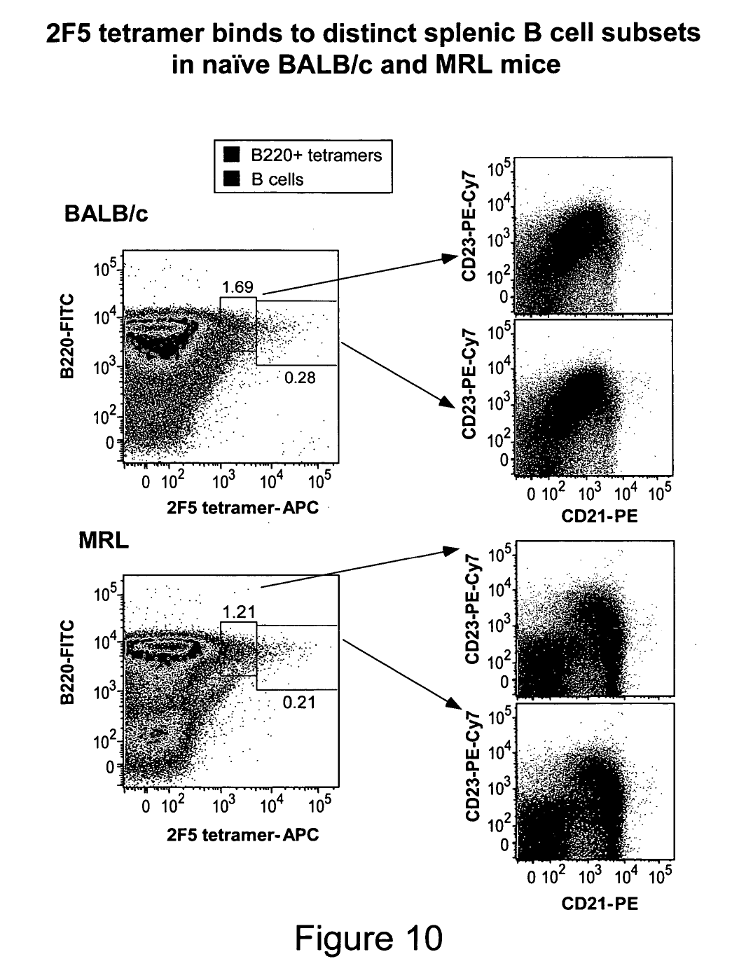

FIG. 7 shows the design of B cell tetramers with the HIV peptide epitope biotinylated and bound to the streptavidin tetramer. The streptavidin can be labled with a number of fluorochromes. FIG. 8 shows that tetramers will cross link B cell Ig receptors on the surface of the B cell. FIG. 9 shows in normal and MRL naive unimmunized mice that there is a B220+ hi population of B cells that bind the tetramer. These data suggest that these cells in normal mice are anergic and do not make antibody constitutively to the 2F5 epitope, while these B cells in MRL are not anergic and make the antibody. FIG. 10 shows the origin of the 2F5+B cells in BALB/c normal and MRL autoimmune mice. The anergic 2F5+B cells in normal mice are B 2 mature follicular cells that are CD23 hi and CD21hi, while the 2F5+B cells in MRL mice are marginal zone B cells and are CD23 low, CD21 hi cells.

The data presented imply that spontaneous anti-MPER antibodies are made in MRL mice because 2F5 epitope-reactive B cells are in different subsets and are under different immunoregulatory controls in naïve BALB/c versus MRL mice.

All references and other information sources cited above are hereby incorporated by reference.

TABLE 1
Neutralization Titers of Guinea pigs Immunized with HIV-1 subtype A, B, C and Group M Consensu (CON-S) ENV Immunogens.

| | 92RWO20 (Subtype A) | | | | JRFL (Subtype B) | | | | 97ZA012 (Subtype C) | | | | CON-S gp140CFI | | | |
|---|---|---|---|---|---|---|---|---|---|---|---|---|---|---|---|---|
| HIV-1 Isolate | Guinea pig Number | | | | Guinea pig Number | | | | Guinea pig Number | | | | Guinea pig Number | | | |
| (Subtype) | 854 | 855 | 856 | 857 | 791 | 793 | 796 | 797 | 862 | 863 | 864 | 865 | 776 | 777 | 778 | 780 |
| BX08 | <20 | <20 | <20 | <20 | 23 | 22 | <20 | <20 | <20 | <20 | <20 | <20 | 1,196 | 412 | 4,856 | 1,817 |
| QH0692 (B) | 34 | <20 | <20 | 36 | 108 | <20 | <20 | <20 | <20 | <20 | <20 | <20 | 109 | <20 | <20 | <20 |
| SS1196(B) | 115 | 83 | 100 | 150 | >540 | >540 | 506 | 489 | 23 | 27 | <20 | <20 | 796 | 296 | 1,339 | 423 |
| SF162 | >540 | 412 | >540 | >540 | >540 | >540 | 92 | 290 | 128 | 421 | 88 | 106 | >540 | >540 | >540 | >540 |
| JRFL(B) | <20 | <20 | <20 | <20 | <20 | <20 | <20 | <20 | <20 | <20 | <20 | <20 | <20 | <20 | <20 | <20 |
| BG1168(B) | <20 | <20 | <20 | <20 | <20 | <20 | <20 | <20 | <20 | <20 | <20 | <20 | <20 | <20 | <20 | <20 |
| 3988(B) | <20 | <20 | <20 | <20 | <20 | <20 | <20 | <20 | <20 | <20 | <20 | <20 | <20 | <20 | <20 | <20 |
| 6101(B) | <20 | <20 | <20 | <20 | <20 | <20 | <20 | <20 | <20 | <20 | <20 | <20 | <20 | <20 | <20 | <20 |
| TV-1(C) | 540 | 443 | 449 | >540 | <20 | <20 | <20 | <20 | 93 | 148 | <20 | <20 | 1,339 | 770 | 2,442 | 724 |
| DU123(C) | 41 | <20 | 48 | 37 | <20 | <20 | <20 | <20 | <20 | 115 | <20 | <20 | 176 | 329 | 387 | 378 |
| DU172(C) | <20 | <20 | <20 | <20 | <20 | <20 | <20 | <20 | <20 | <20 | <20 | <20 | <20 | 235 | <20 | 213 |
| ZM18108.6(C) | <20 | <20 | <20 | <20 | <20 | <20 | <20 | <20 | <20 | <20 | <20 | <20 | 84 | 61 | 86 | 43 |
| 92BR025.9(C) | 403 | 168 | 258 | 311 | <20 | <20 | <20 | <20 | 55 | 50 | <20 | 39 | 1,819 | 1,408 | 3,207 | 1,336 |
| ZM14654.7(C) | <20 | <20 | <20 | 27 | 23 | 22 | <20 | <20 | 21 | 22 | <20 | <20 | <20 | 33 | 30 | <20 |
| 96ZM651(C) | <20 | <20 | <20 | <20 | <20 | <20 | <20 | <20 | <20 | <20 | <20 | <20 | <20 | 22 | <20 | <20 |
| DU151(C) | <20 | <20 | <20 | <20 | <20 | <20 | <20 | <20 | <20 | <20 | <20 | <20 | <20 | <20 | <20 | <20 |
| 97ZA012(C) | <20 | <20 | <20 | <20 | <20 | <20 | 36 | 20 | <20 | <20 | <20 | <20 | <20 | <20 | <20 | <20 |
| DU422(C) | <20 | <20 | <20 | <20 | <20 | <20 | <20 | <20 | <20 | <20 | <20 | <20 | <20 | <20 | <20 | <20 |
| DU156(C) | <20 | <20 | <20 | <20 | <20 | <20 | <20 | <20 | <20 | <20 | <20 | <20 | <20 | <20 | <20 | <20 |
| 92RWO20(A) | 150 | 71 | 100 | 106 | <20 | <20 | <20 | <20 | <20 | <20 | <20 | <20 | 116 | 204 | 95 | 177 |

*.50% Neutralization titers of serum after 4th or 5th immunizations. Neutralization was considered positive (number in bold) if the titer of post-immune serum minus the titer of pre-immune bleed serum was >30 and the post-immune titer was at least 3× over the pre-immune titer. In addition, anti-CON-S sera, No. 776, 777, 778 and 780 were assayed against additional 10 subtype A isolates (----) and were negative).

What is claimed is:

1. A method of screening an immunogen comprising: (i) administering said immunogen to a normal mammal and to a mammal of the same species having a defect in B cell tolerance, (ii) obtaining an antibody-containing sample from said normal mammal and from said mammal having a defect in B cell tolerance, and (ii) assaying said samples for the presence of antibodies against said immunogen,
wherein the presence of antibodies against said immunogen in said sample from said mammal having a defect in B cell tolerance but not in said sample from said normal mammal indicates that said immunogen is structurally correct and that antibodies are not made by said normal mammal as a result of host control, and
wherein the absence of antibodies against said immunogen in said sample from said mammal having a defect in B cell tolerance and in said sample from said normal mammal indicates that said immunogen is structurally incorrect or is not exposed.

2. The method according to claim 1 wherein said mammal having a defect in B cell tolerance is an autoimmune mammal.

3. The method according to claim 1 wherein said mammals are rodents.

4. The method according to claim 3 wherein said rodents are mice.

5. The method according to claim 4 wherein said autoimmune mice are MRL/lpr^{-/-} mice.

6. The method according to claim 1 wherein said samples are serum samples.

7. A method of screening an immunogen comprising: (i) administering said immunogen to a normal mammal and to an mammal of the same species lacking T regulatory cells or lacking T regulatory cell function, (ii) obtaining an antibody-containing sample from said normal mammal and said mammal lacking T regulatory cells or lacking T regulatory cell function, and (ii) assaying said samples for the presence of antibodies against said immunogen,
wherein the presence of antibodies against said immunogen in said sample from said mammal lacking T regulatory cells or lacking T regulatory cell function but not in said sample from said normal mammal indicates that said immunogen is structurally correct and that antibodies are not made by said normal mammal as a result of host control, and
wherein the absence of antibodies against said immunogen in said sample from said mammal lacking T regulatory cells or T regulatory cell function and in said sample from said normal mammal indicates that said immunogen is structurally incorrect or is not exposed.

8. The method according to claim 7 wherein said mammals are rodents.

9. The method according to claim 8 wherein said rodents are mice.

10. The method according to claim 8 wherein said rodent lacking T regulatory cells or T regulatory cell function is a rodent that lacks T regulatory cells as a result of neonatal thymectomy.

11. The method according to claim 7 wherein said samples are serum samples.

12. The method according to claim 1 wherein said immunogen is from an infectious agent.

13. The method according to claim 12 wherein said infectious agent is a virus.

14. The method according to claim 13 wherein said virus is HIV, Hepatitis C, West Nile Virus, or Ebola Hemmorhagic Fever Virus.

15. The method according to claim 14 wherein said virus is HIV and said immunogen is an HIV tat protein immunogen or and HIV-1 envelope protein immunogen.

16. The method according to claim 7 wherein said immunogen is from an infectious agent.

17. The method according to claim 16 wherein said infectious agent is a virus.

18. The method according to claim 17 wherein said virus is HIV, Hepatitis C, West Nile Virus, or Ebola Hemmorhagic Fever Virus.

19. The method according to claim 18 wherein said virus is HIV and said immunogen is an HIV tat protein immunogen or and HIV-1 envelope protein immunogen.
